(19) United States
(12) Patent Application Publication
(10) Pub. No.: US 2005/0224756 A1
(43) Pub. Date: Oct. 13, 2005

(54) 3,3,4,4-Tetrafluorocyclopentane compounds, used as components of liquid crystal media

(76) Inventors: Volker Reiffenrath, Rossdorf (DE); Georg Lussem, Ober-Ramstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/518,925

(22) PCT Filed: Jun. 18, 2003
(86) PCT No.: PCT/EP03/06447

§ 371 Date: Dec. 23, 2004

(30) Foreign Application Priority Data
Jun. 24, 2002 (DE) 10228183.1

(51) Int. Cl. C09K 19/34; C09K 19/30; C09K 19/32; C07C 25/13; C07C 23/08

(52) U.S. Cl. 252/299.61; 252/299.63; 570/130; 252/299.62

(57) ABSTRACT

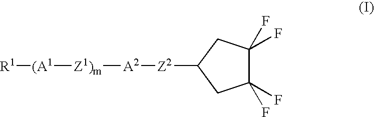

The invention relates to 3,3,4,4-tetrafluorocyclopentane compounds of formula (I), in which R¹, A¹, A², Z¹, Z^{2 m are defined as cited in claim} 1. Said compounds are suitable for use as components of liquid crystal media.

The invention relates to 3,3,4,4-tetrafluorocyclopentane compounds of the formula I 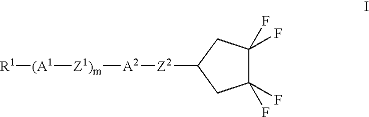
in which
R¹ is H, or an alkyl radical having up to 15 carbon atoms which is unsubstituted, monosubstituted by CN or CF₃ or at least monosubstituted by halogen, where, in addition, one or more CH₂ groups in these radicals may each, independently of one another, be replaced by —O—, —CH═CH—, —S—, 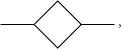
   —CO—, —CO—O—, —O—CO— or —O—CO—O— in such a way that O atoms are not linked directly to one another,
A¹ and A² are each, independently of one another,
   (a) a trans-1,4-cyclohexylene radical, in which, in addition, one or more non-adjacent CH₂ groups may be replaced by —O— and/or —S—,
   (b) a 1,4-phenylene radical, in which, in addition, one or two CH groups may be replaced by N,
   (c) 1,4-cyclohexenylene,
   (d) a radical from the group consisting of 1,4-bicyclo[2.2.2]-octylene, piperidine-1,4-diyl, naphthalene-2,6-diyl, decahydronaphthalene-2,6-diyl and 1,2,3,4-tetrahydronaphthalene-2,6-diyl,
      where the radicals (a) to (d) may be substituted by one or more, in particular one or two, fluorine atoms,
Z¹ and Z² are each, independently of one another, —CO—O—, —O—CO—, —CH₂O—, —OCH₂—, —CH₂CH₂—, —CH═CH—, —C≡C—, —(CH₂)₄—, —CF₂O—, —OCF₂—, —C₂F₄—, —CH═CH—CH₂CH₂— or a single bond,
m is 0, 1 or 2.

The invention furthermore relates to the use of these compounds as components of liquid-crystalline media and to liquid-crystal and electro-optical display elements which contain the liquid-crystalline media according to the invention.

The compounds of the formula I can be used as components of liquid-crystalline media, in particular for displays based on the principle of the twisted cell, the guest-host effect, the effect of deformation of aligned phases or the effect of dynamic scattering.

The invention had the object of finding novel stable liquid-crystalline or mesogenic compounds which are suitable as components of liquid-crystalline media and in particular at the same time have comparatively low viscosity and relatively high dielectric anisotropy.

In view of the very wide variety of areas of application of such compounds having high Δε, however, it was desirable to have available further compounds having high nematogeneity which have properties precisely customised to the particular applications.

It has now been found that compounds of the formula I are eminently suitable as components of liquid-crystalline media. In particular, they have comparatively low viscosities. The compounds according to the invention can be used to prepare mixtures having a very low Δn, high positive As and a very high voltage holding ratio. Mixtures of this type are particularly suitable for reflective applications. They can be used to obtain stable liquid-crystalline media having a broad mesophase range and advantageous values for the optical anisotropy (Δn) and dielectric anisotropy (Δε). Furthermore, the media according to the invention have very good low-temperature behaviour.

In addition, the provision of compounds of the formula I very generally considerably broadens the range of liquid-crystalline substances which are suitable, from various applicational points of view, for the preparation of liquid-crystalline mixtures.

The compounds of the formula I have a broad range of applications. Depending on the choice of substituents, these compounds can serve as base materials of which liquid-crystalline media are predominantly composed; however, it is also possible to add compounds of the formula I to liquid-crystalline base materials from other classes of compound in order, for example, to modify the dielectric and/or optical anisotropy of a dielectric of this type and/or to optimise its threshold voltage and/or its viscosity.

In the pure state, the compounds of the formula I are colourless and form liquid-crystalline mesophases in a temperature range which is favourably located for electro-optical use. They are stable chemically, thermally and to light.

The invention thus relates to the compounds of the formula I and to the use of these compounds as components of liquid-crystalline media. The invention furthermore relates to liquid-crystalline media comprising at least one compound of the formula I and to liquid-crystal display elements, in particular electro-optical display elements, which contain such media.

For reasons of simplicity, A³ below is a radical of the formula 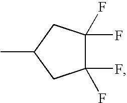
Cyc is a 1,4-cyclohexyl radical, Che is a 1,4-cyclohexenylene radical, Dio is a 1,3-dioxane-2,5-diyl radical, Dit is a 1,3-dithiane-2,5-diyl radical, Phe is a 1,4-phenylene radical, Pyd is a pyridine-2,5-diyl radical, Pyr is a pyrimidine-2,5-diyl radical, and Bi is a bicyclo[2.2.2]-octylene radical, where Cyc and/or Phe may be unsubstituted or monosubstituted or disubstituted by F.

A¹ and A² are preferably selected from the group consisting of Cyc, Che, Phe, Pyr, Pyd and Dio.

The compounds of the formula I accordingly include bicyclic compounds of the sub-formulae Ia and Ib:
R¹-A²-A³ Ia
R¹-A²-Z²-A³ Ib
tricyclic compounds of the sub-formulae Ic to Ie:
R¹-A¹-A²-A³ Ic
R¹-A¹-Z¹-A²-A³ Id
R¹-A¹-A²-Z²-A³ Ie
and tetracyclic compounds of the sub-formulae If to Ii:
R¹-A¹-A¹-A²-A³ If
R¹-A¹-Z¹-A¹-A²-A³ Ig
R¹-A¹-A¹-A²-Z²-A³ Ih
R¹-A¹-Z¹-A¹-A²-Z²-A³ Ii

Preference is also given to compounds of the formula I and of all sub-formulae in which A¹ and/or A² is 1,4-phenylene which is monosubstituted or disubstituted by F. These are, in particular, 2-fluoro-1,4-phenylene, 3-fluoro-1,4-phenylene and 3,5-difluoro-1,4-phenylene. A¹ and A² are preferably 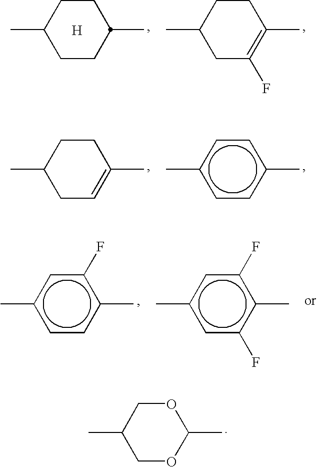
Z¹ and Z² are preferably a single bond, —CO—O—, —O—CO—, —CF₂O— and —CH₂CH₂—, secondarily preferably —OCF₂—, —CH₂O— and —OCH₂—. If one of the radicals Z¹ and Z² is —(CH₂)₄— or —CH═CH—CH₂CH₂—, the other radical Z¹ or Z² (if present) is preferably a single bond.
R¹ is preferably alkyl, alkoxy, alkenyl or alkenyloxy having up to 7 carbon atoms.
m is preferably 0 or 1.

If R¹ is an alkyl radical and/or an alkoxy radical, this may be straight-chain or branched. It is preferably straight-chain, has 1, 2, 3, 5, 6 or 7 carbon atoms and accordingly is preferably methyl, ethyl, propyl, pentyl, heptyl, butyl, hexyl, ethoxy, propoxy, butoxy, pentoxy, hexyloxy or heptyloxy, furthermore octyl, nonyl, decyl, undecyl, dodecyl, tridecyl, tetradecyl, pentadecyl, methoxy, octyloxy, nonyloxy, decyloxy, undecyloxy, dodecyloxy, tridecyloxy or tetradecyloxy.

Oxaalkyl is preferably straight-chain 2-oxapropyl (=methoxymethyl), 2-(=ethoxymethyl) or 3-oxabutyl (=2-methoxyethyl), 2-, 3- or 4-oxapentyl, 2-, 3-, 4- or 5-oxahexyl, 2-, 3-, 4-, 5- or 6-oxaheptyl, 2-, 3-, 4-, 5-, 6- or 7-oxaoctyl, 2-, 3-, 4-, 5-, 6-, 7- or 8-oxanonyl, or 2-, 3-, 4-, 5-, 6-, 7-, 8- or 9-oxadecyl.

If R¹ is an alkyl radical in which one CH₂ group has been replaced by —CH═CH—, this may be straight-chain or branched. It is preferably straight-chain and has from 2 to 10 carbon atoms. Accordingly, it is in particular vinyl, prop-1- or -2-enyl, but-1-, -2- or -3-enyl, pent-1-, -2-, -3- or -4-enyl, hex-1-, -2-, -3-, -4- or -5-enyl, hept-1-, -2-, -3-, -4-, -5- or -6-enyl, oct-1-, -2-, -3-, -4-, -5-, -6- or -7-enyl, non-1-, -2-, -3-, -4-, -5-, -6-, -7- or -8-enyl, or dec-1-, -2-, -3-, 4-, -5-, -6-, -7-, -8- or -9-enyl.

If R¹ is an alkyl radical in which one CH₂ group has been replaced by —O— and one has been replaced by —CO—, these are preferably adjacent. These thus contain an acyloxy group —CO—O— or an oxycarbonyl group —O—CO—. These are preferably straight-chain and have from 2 to 6 carbon atoms. Accordingly, they are in particular acetoxy, propionyloxy, butyryloxy, pentanoyloxy, hexanoyloxy, acetoxymethyl, propionyloxymethyl, butyryloxymethyl, pentanoyloxymethyl, 2-acetoxyethyl, 2-propionyloxyethyl, 2-butyryloxyethyl, 3-acetoxypropyl, 3-propionyloxypropyl, 4-acetoxybutyl, methoxycarbonyl, ethoxycarbonyl, propoxycarbonyl, butoxycarbonyl, pentoxycarbonyl, methoxycarbonylmethyl, ethoxycarbonylmethyl, propoxycarbonylmethyl, butoxycarbonylmethyl, 2-(methoxycarbonyl)ethyl, 2-(ethoxycarbonyl)ethyl, 2-(propoxycarbonyl)ethyl, 3-(methoxycarbonyl)-propyl, 3-(ethoxycarbonyl)propyl or 4-(methoxycarbonyl)butyl.

If R¹ is an alkyl radical in which one CH₂ group has been replaced by unsubstituted or substituted —CH═CH— and an adjacent CH₂ group has been replaced by CO or CO—O or O—CO, this may be straight-chain or branched. It is preferably straight-chain and has from 4 to 13 carbon atoms. Accordingly, it is in particular acryloyloxymethyl, 2-acryloyloxyethyl, 3-acryloyloxypropyl, 4-acryloyloxybutyl, 5-acryloyloxypentyl, 6-acryloyloxyhexyl, 7-acryloyloxyheptyl, 8-acryloyloxyoctyl, 9-acryloyloxynonyl, 10-acryloyloxydecyl, methacryloyloxymethyl, 2-methacryloyloxyethyl, 3-methacryloyloxypropyl, 4-methacryloyloxybutyl, 5-methacryloyloxypentyl, 6-methacryloyloxyhexyl, 7-methacryloyloxyheptyl, 8-methacryloyloxyoctyl or 9-methacryloyloxynonyl.

If R¹ is an alkyl or alkenyl radical which is monosubstituted by CN or CF₃, this radical is preferably straight-chain and the substitution by CN or CF₃ is in the ω-position.

If R¹ is an alkyl or alkenyl radical which is at least monosubstituted by halogen, this radical is preferably straight-chain, and halogen is preferably F or Cl. In the case of polysubstitution, halogen is preferably F. The resultant radicals also include perfluorinated radicals. In the case of monosubstitution, the fluorine or chlorine substituent may be in any desired position, but is preferably in the ω-position.

Compounds of the formula I which have wing groups R¹ which are suitable for polymerisation reactions are suitable for the preparation of liquid-crystalline polymers.

Compounds of the formula I containing branched wing groups R may occasionally be of importance owing to better solubility in the conventional liquid-crystalline base materials, but in particular as chiral dopants if they are optically active. Smectic compounds of this type are suitable as components of ferroelectric materials.

Compounds of the formula I having S_{A} phases are suitable, for example, for thermally addressed displays.

Branched groups of this type generally contain not more than one chain branch. Preferred branched radicals R are isopropyl, 2-butyl (=1-methylpropyl), isobutyl (=2-methylpropyl), 2-methylbutyl, isopentyl (=3-methylbutyl), 2-methylpentyl, 3-methylpentyl, 2-ethylhexyl, 2-propylpentyl, isopropoxy, 2-methylpropoxy, 2-methylbutoxy, 3-methylbutoxy, 2-methylpentoxy, 3-methylpentoxy, 2-ethylhexyloxy, 1-methylhexyloxy and 1-methylheptyloxy.

If R¹ is an alkyl radical in which two or more CH₂ groups have been replaced by —O— and/or —CO—O—, this may be straight-chain or branched. It is preferably branched and has from 3 to 12 carbon atoms. Accordingly, it is in particular biscarboxymethyl, 2,2-biscarboxyethyl, 3,3-biscarboxypropyl, 4,4-biscarboxybutyl, 5,5-biscarboxypentyl, 6,6-biscarboxyhexyl, 7,7-biscarboxyheptyl, 8,8-biscarboxyoctyl, 9,9-biscarboxynonyl, 10,10-biscarboxydecyl, bis(methoxycarbonyl)methyl, 2,2-bis(methoxycarbonyl)ethyl, 3,3-bis(methoxycarbonyl)propyl, 4,4-bis(methoxycarbonyl)butyl, 5,5-bis(methoxycarbonyl)pentyl, 6,6-bis(methoxycarbonyl)hexyl, 7,7-bis(methoxycarbonyl)heptyl, 8,8-bis(methoxycarbonyl)octyl, bis(ethoxycarbonyl)methyl, 2,2-bis(ethoxycarbonyl)ethyl, 3,3-bis(ethoxycarbonyl)propyl, 4,4-bis(ethoxycarbonyl)butyl or 5,5-bis(ethoxycarbonyl)hexyl.

Compounds of the formula I which have wing groups R¹ which are suitable for polycondensations are suitable for the preparation of liquid-crystalline polycondensates.

The formula I covers both the racemates of these compounds and the optical antipodes, and mixtures thereof.

Of these compounds of the formula I and the sub-formulae, preference is given to those in which at least one of the radicals present therein has one of the preferred meanings indicated.

In the compounds of the formula I, preference is given to the stereoisomers in which the Cyc and piperidine rings are trans-1,4-disubstituted. Those of the above-mentioned formulae which contain one or more Pyd, Pyr and/or Dio groups in each case cover the two 2,5-positional isomers.

Preferred smaller groups of compounds are those of the sub-formulae I1 to I30:

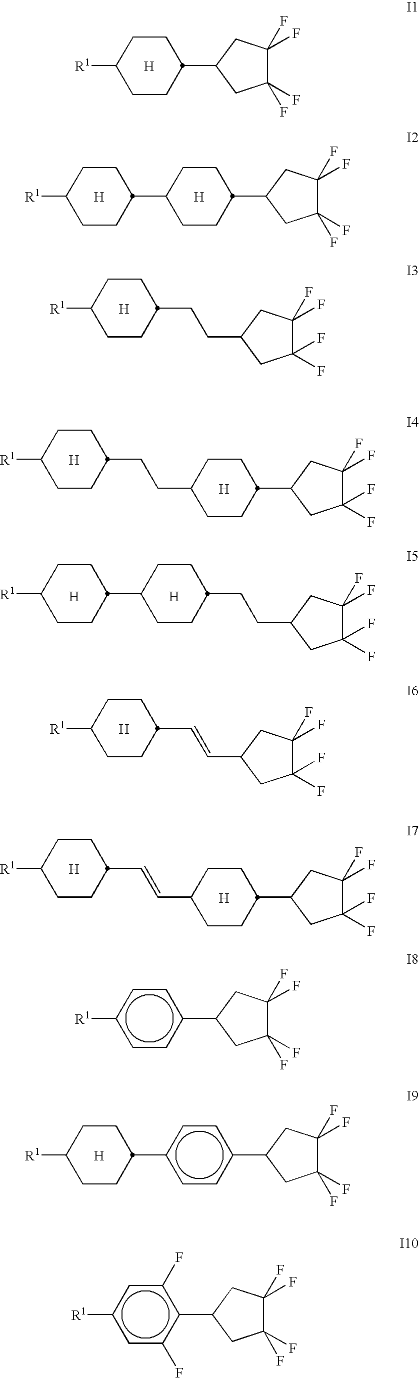

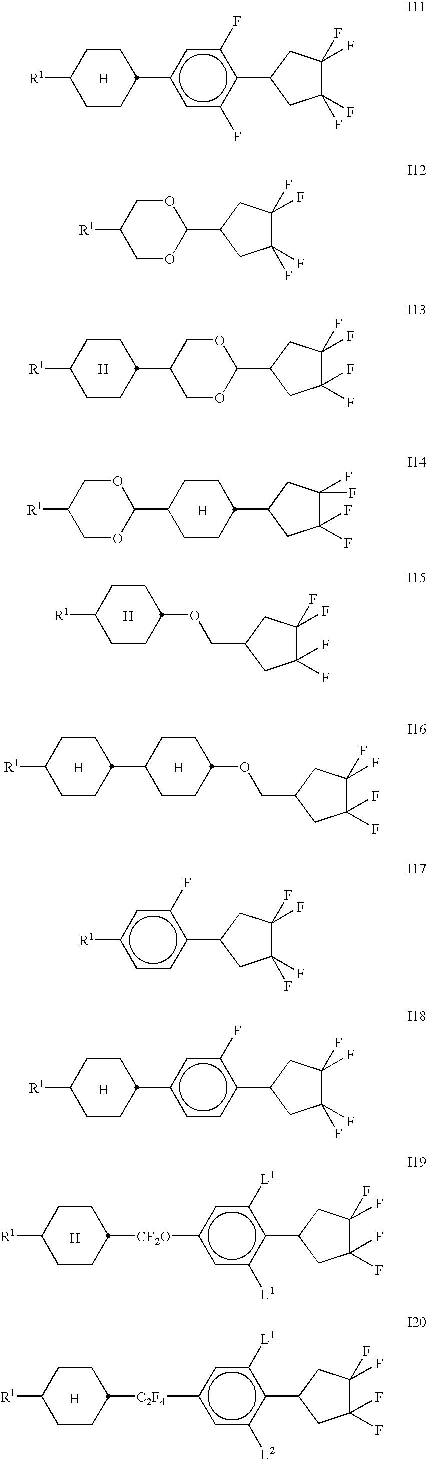

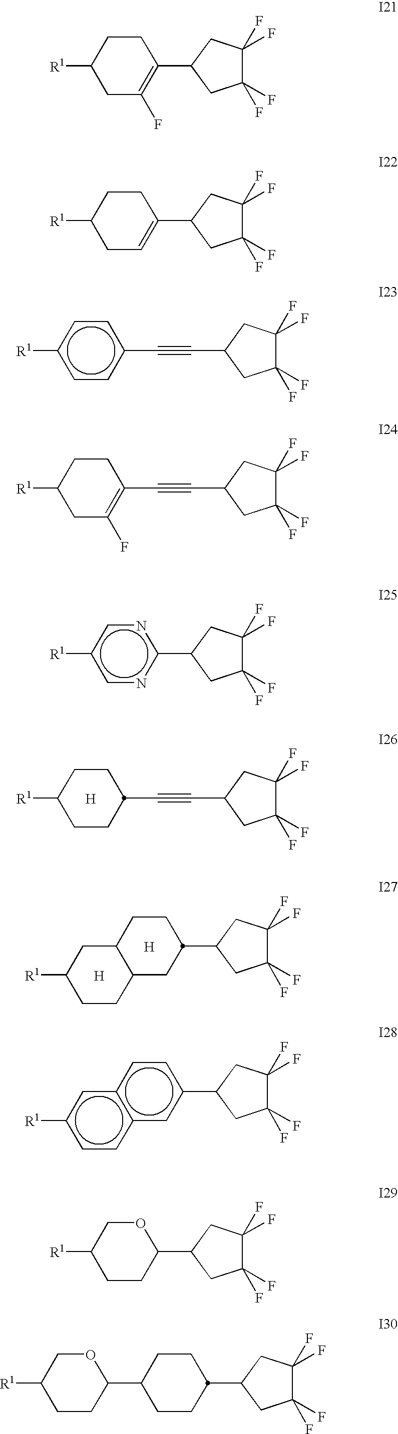

The 1,4-cyclohexenylene group preferably has the following structures:

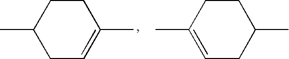

The compounds of the formula I are prepared by methods known per se, as described in the literature (for example in the standard works, such as Houben-Weyl, Methoden der Organischen Chemie [Methods of Organic Chemistry], Georg-Thieme-Verlag, Stuttgart), to be precise under reaction conditions which are known and suitable for the said reactions.

Use can also be made here of variants which are known per se, but are not mentioned here in greater detail.

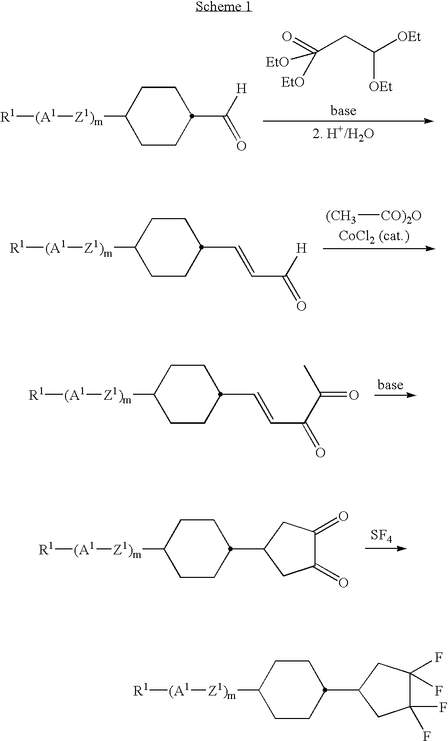

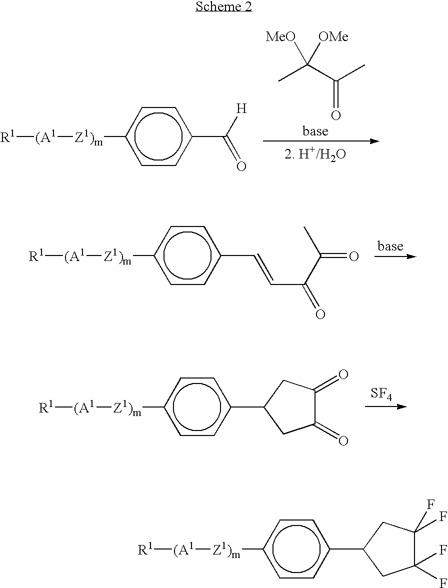

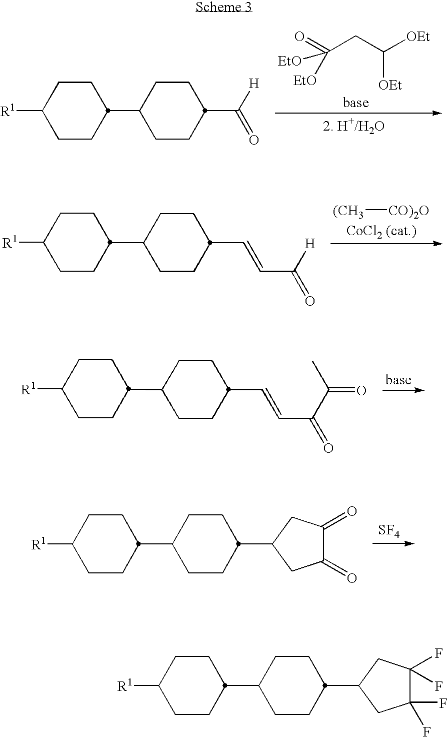

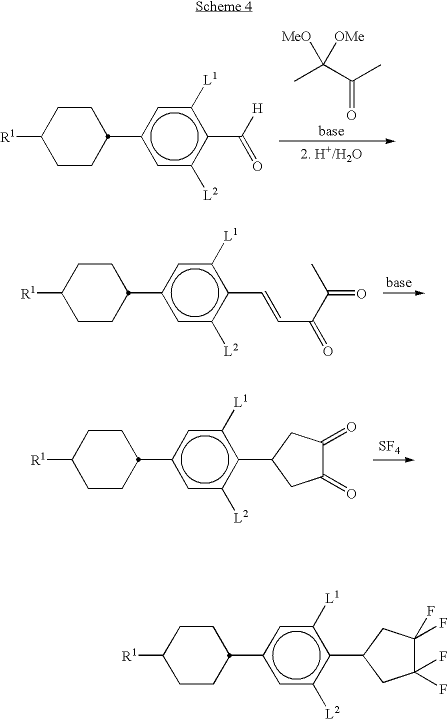

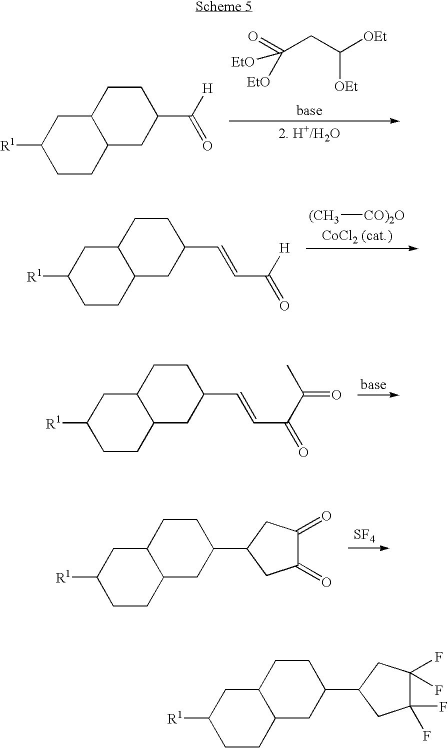

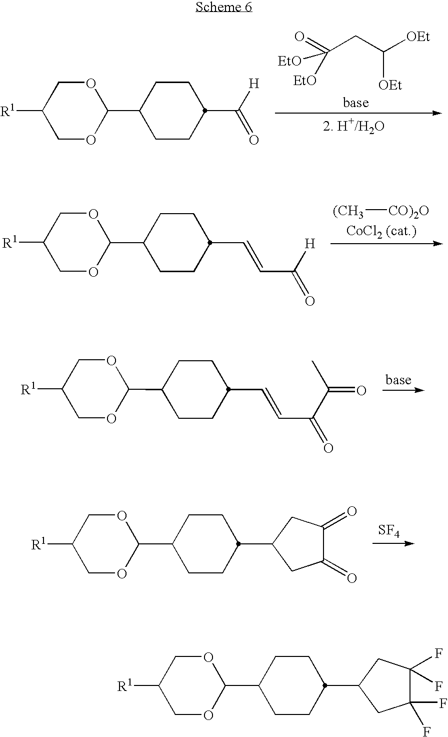

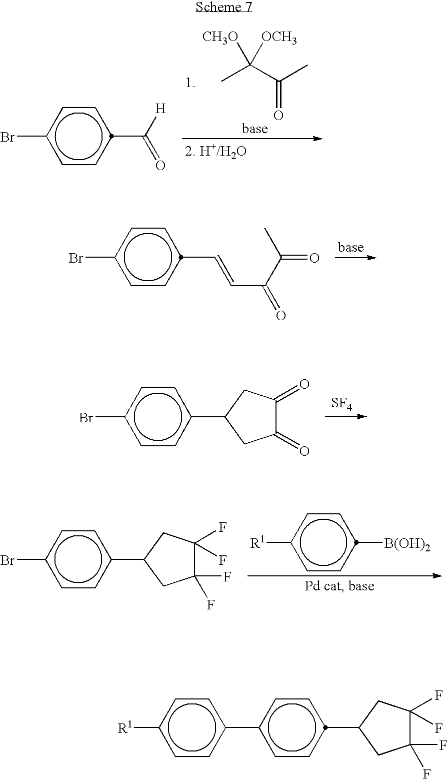

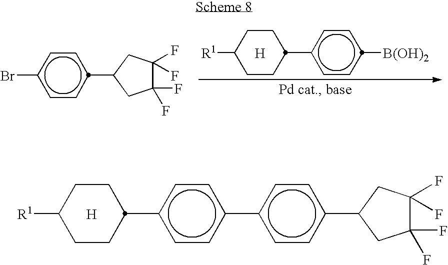

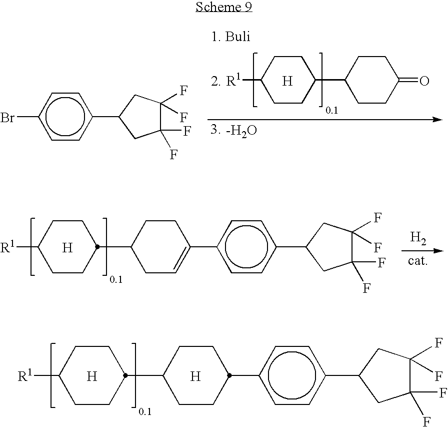

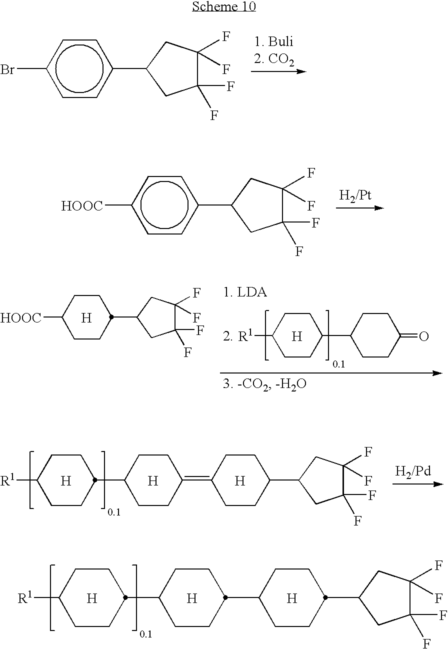

The liquid-crystalline media according to the invention preferably comprise from 2 to 40, in particular from 4 to 30, components as further constituents besides one or more compounds according to the invention. These media very particularly preferably comprise from 7 to 25 components besides one or more compounds according to the invention. These further constituents are preferably selected from nematic or nematogenic (monotropic or isotropic) substances, in particular substances from the classes of the azoxybenzenes, benzylideneanilines, biphenyls, terphenyls, phenyl or cyclohexyl benzoates, phenyl or cyclohexyl esters of cyclohexanecarboxylic acid, phenyl or cyclohexyl esters of cyclohexylbenzoic acid, phenyl or cyclohexyl esters of cyclohexylcyclohexanecarboxylic acid, cyclohexylphenyl esters of benzoic acid, of cyclohexanecarboxylic acid or of cyclohexylcyclohexanecarboxylic acid, phenylcyclohexanes, cyclohexylbiphenyls, phenylcyclohexylcyclohexanes, cyclohexylcyclohexanes, cyclohexylcyclohexylcyclohexenes, 1,4-biscyclohexylbenzenes, 4,4′-biscyclohexylbiphenyls, phenyl- or cyclohexylpyrimidines, phenyl- or cyclohexylpyridines, phenyl- or cyclohexyldioxanes, phenyl- or cyclohexyl-1,3-dithianes, 1,2-diphenylethanes, 1,2-dicyclohexylethanes, 1-phenyl-2-cyclohexylethanes, 1-cyclohexyl-2-(4-phenylcyclohexyl)ethanes, 1-cyclohexyl-2-biphenylylethanes, 1-phenyl-2-cyclohexylphenylethanes, optionally halogenated stilbenes, benzyl phenyl ethers, tolans and substituted cinnamic acids. The 1,4-phenylene groups in these compounds may also be fluorinated.

The most important compounds suitable as further constituents of media according to the invention can be characterised by the formulae 1, 2, 3, 4 and 5:

| | |
|---|---|
| R′-L-E-R″ | 1 |
| R′-L-COO-E-R″ | 2 |
| R′-L-OOC-E-R″ | 3 |
| R′-L-CH₂CH₂-E-R″ | 4 |
| R′-L-C≡C-E-R″ | 5 |

In the formulae 1, 2, 3, 4 and 5, L and E, which may be identical or different, are each, independently of one another, a divalent radical from the group formed by -Phe-, -Cyc-, -Phe-Phe-, -Phe-Cyc-, -Cyc-Cyc-, -Pyr-, -Dio-, -G-Phe- and -G-Cyc- and their mirror images, where Phe is unsubstituted or fluorine-substituted 1,4-phenylene, Cyc is trans-1,4-cyclohexylene or 1,4-cyclohexenylene, Pyr is pyrimidine-2,5-diyl or pyridine-2,5-diyl, Dio is 1,3-dioxane-2,5-diyl and G is 2-(trans-1,4-cyclohexyl)ethyl, Pyr is pyrimidine-2,5-diyl, Pyd is pyridine-2,5-diyl or Py is a pyran ring.

One of the radicals L and E is preferably Cyc, Phe or Pyr. E is preferably Cyc, Phe or Phe-Cyc. The media according to the invention preferably comprise one or more components selected from the compounds of the formulae 1, 2, 3, 4 and 5 in which L and E are selected from the group consisting of Cyc, Phe, Py and Pyr and simultaneously one or more components selected from the compounds of the formulae 1, 2, 3, 4 and 5 in which one of the radicals L and E is selected from the group consisting of Cyc, Phe and Pyr and the other radical is selected from the group consisting of -Phe-Phe-, -Phe-Cyc-, -Cyc-Cyc-, -G-Phe- and -G-Cyc-, and optionally one or more components selected from the compounds of the formulae 1, 2, 3, 4 and 5 in which the radicals L and E are selected from the group consisting of -Phe-Cyc-, -Cyc-Cyc-, -G-Phe- and -G-Cyc-.

In a smaller sub-group of the compounds of the formulae 1, 2, 3, 4 and 5, R′ and R″ are each, independently of one another, alkyl, alkenyl, alkoxy, alkoxyalkyl, alkenyloxy or alkanoyloxy having up to 8 carbon atoms. This smaller sub-group is called group A below, and the compounds are referred to by the sub-formulae 1a, 2a, 3a, 4a and 5a. In most of these compounds, R′ and R″ are different from one another, one of these radicals usually being alkyl, alkenyl, alkoxy or alkoxyalkyl.

In another smaller sub-group of the compounds of the formulae 1, 2, 3, 4 and 5, which is known as group B, R″ is —F, —Cl, —NCS or —(O)ᵢCH₃₋₍ₖ₊₁₎FₖCl_{I}, where i is 0 or 1, and k+1 is 1, 2 or 3; the compounds in which R″ has this meaning are referred to by the sub-formulae 1b, 2b, 3b, 4b and 5b. Particular preference is given to those compounds of the sub-formulae 1b, 2b, 3b, 4b and 5b in which R″ is —F, —Cl, —NCS, —CF₃, —OCHF₂ or —OCF₃.

In the compounds of the sub-formulae 1b, 2b, 3b, 4b and 5b, R′ is as defined for the compounds of the sub-formulae 1a-5a and is preferably alkyl, alkenyl, alkoxy or alkoxyalkyl.

In a further smaller sub-group of the compounds of the formulae 1, 2, 3, 4 and 5, R″ is —CN; this sub-group is referred to below as group C, and the compounds of this sub-group are correspondingly described by subformulae 1c, 2c, 3c, 4c and 5c. In the compounds of the sub-formulae 1c, 2c, 3c, 4c and 5c, R′ is as defined for the compounds of the sub-formulae 1a-5a and is preferably alkyl, alkoxy or alkenyl.

Besides the preferred compounds of groups A, B and C, other compounds of the formulae 1, 2, 3, 4 and 5 having other variants of the proposed substituents are also customary. All these substances are obtainable by methods which are known from the literature or analogously thereto.

Besides compounds of the formula I according to the invention, the media according to the invention preferably comprise one or more compounds selected from group A and/or group B and/or group C. The proportions by weight of the compounds from these groups in the media according to the invention are preferably

| R¹ | —(A¹-Z¹)ₘ— |
|---|---|
| n-C₇H₁₅ | 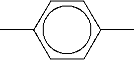 |
| CH₂═CH | 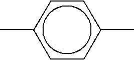 |
| CH₃—CH═CH₂ | 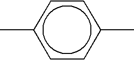 |
| CH₂═CHCH₂CH₂ | 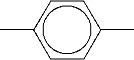 |
| CH₃CH═CHCH₂CH₂ | 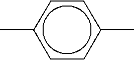 |
| CH₃ | 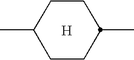 |
| C₂H₅ | 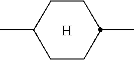 |
| n-C₃H₇ | 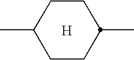 |
| n-C₅H₁₁ | 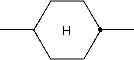 |
| CH₃ | 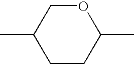 |
| C₂H₅ | 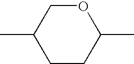 |
| n-C₃H₇ | 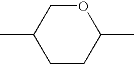 |
| n-C₅H₁₁ | 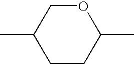 |

group A: from 0 to 90%, preferably from 20 to 90%, in particular from 30 to 90%
group B: from 0 to 80%, preferably from 10 to 80%, in particular from 10 to 65%
group C: from 0 to 80%, preferably from 5 to 80%, in particular from 5 to 50%

where the sum of the proportions by weight of the group A and/or B and/or C compounds present in the respective media according to the invention is preferably from 5 to 90% and in particular from 10 to 90%.

The media according to the invention preferably comprise from 1 to 40%, particularly preferably from 5 to 30%, of compounds according to the invention. Preference is furthermore given to media comprising more than 40%, in particular from 45 to 90%, of compounds according to the invention. The media preferably comprise three, four or five compounds according to the invention.

The media according to the invention are prepared in a manner conventional per se. In general, the components are dissolved in one another, advantageously at elevated temperature. By means of suitable additives, the liquid-crystalline phases in accordance with the invention can be modified in such a way that they can be used in all types of liquid-crystal display elements that have been disclosed hitherto. Additives of this type are known to the person skilled in the art and are described in detail in the literature (H. Kelker/R. Hatz, Handbook of Liquid Crystals, Verlag Chemie, Weinheim, 1980). For example, pleochroic dyes can be added for the preparation of coloured guest-host systems or substances can be added in order to modify the dielectric anisotropy, the viscosity and/or the alignment of the nematic phases. Furthermore, stabilisers and antioxidants can be added.

The mixtures according to the invention are suitable for TN, STN, ECB and IPS applications, in particular for TFT applications.

In the present application and in the examples below, the structures of the liquid-crystal compounds are indicated by means of acronyms, the transformation into chemical formulae taking place in accordance with Tables A and B below. All radicals CₙH₂ₙ₊₁ and CₘH₂ₘ₊₁ are straight-chain alkyl radicals having n and m carbon atoms respectively. The coding in Table B is self-evident. In Table A, only the acronym for the parent structure is indicated. In individual cases, the acronym for the parent structure is followed, separated by a dash, by a code for the substituents R¹, R², L¹ und L²:

| Code for R¹, | | | | |
|---|---|---|---|---|
| R², L¹, L² | R¹ | R² | L¹ | L² |
| nm | CₙH₂ₙ₊₁ | CₘH₂ₘ₊₁ | H | H |
| nOm | CₙH₂ₙ₊₁ | OCₘH₂ₘ₊₁ | H | H |
| nO.m | OCₙH₂ₙ₊₁ | CₘH₂ₘ₊₁ | H | H |
| n | CₙH₂ₙ₊₁ | CN | H | H |
| nN.F | CₙH₂ₙ₊₁ | CN | H | F |
| nF | CₙH₂ₙ₊₁ | F | H | H |
| nOF | OCₙH₂ₙ₊₁ | F | H | H |
| nCl | CₙH₂ₙ₊₁ | Cl | H | H |
| nF.F | CₙH₂ₙ₊₁ | F | H | F |
| nF.F.F | CₙH₂ₙ₊₁ | F | F | F |
| nCF₃ | CₙH₂ₙ₊₁ | CF₃ | H | H |
| nOCF₃ | CₙH₂ₙ₊₁ | OCF₃ | H | H |
| nOCF₂ | CₙH₂ₙ₊₁ | OCHF₂ | H | H |
| nS | CₙH₂ₙ₊₁ | NCS | H | H |
| rVsN | CᵣH₂ᵣ₊₁—CH═CH—CₛH₂ₛ— | CN | H | H |
| rEsN | CᵣH₂ᵣ₊₁—O—CₛH₂ₛ— | CN | H | H |
| nAm | CₙH₂ₙ₊₁ | COOCₘH₂ₘ₊₁ | H | H |

Besides one or more compounds of the formula 1, liquid-crystalline mixtures according to the invention comprise two, three or more components selected from Tables A and B.

TABLE A
(L¹, L², L³ = H or F)

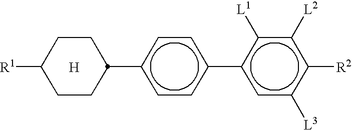

BCH

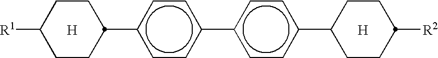

CBC

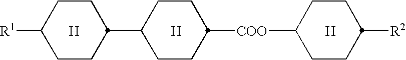

CH

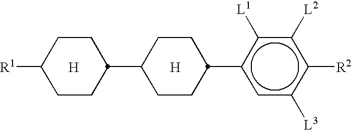

CCP

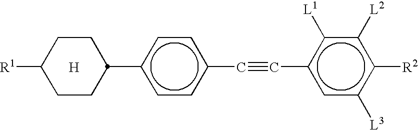

CPTP

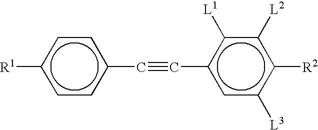

PTP

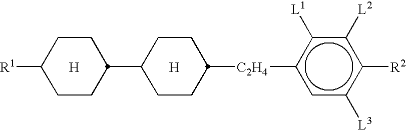

ECCP

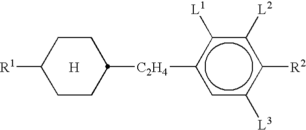

EPCH

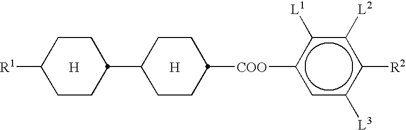

CP

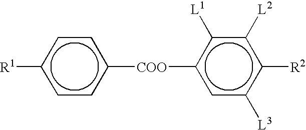

ME

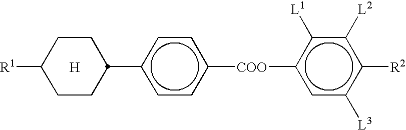

HP

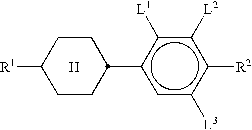

PCH

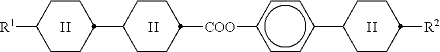

CCPC

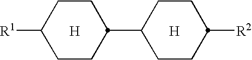

CCH

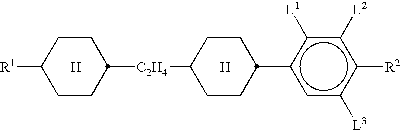

CECP

TABLE B

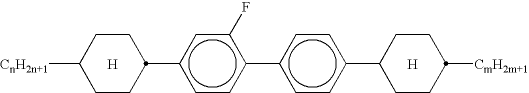

CBC-nmF

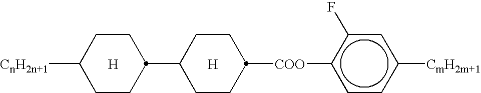

CP-nmF

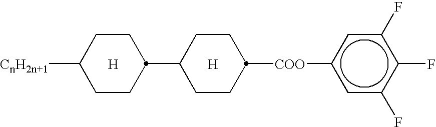

CCZU-n-F

| | |
|---|---|
| 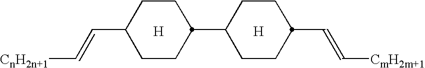 | 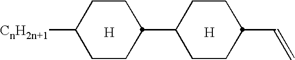 |
| CC-nV-Vm | CC-n-V |
| 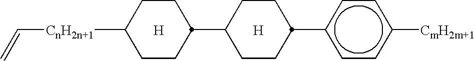 | 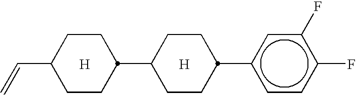 |
| CCP-Vn-m | CCG-V-F |
| 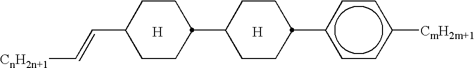 | 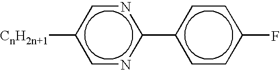 |
| CCP-nV-m | PYP-nF |
| 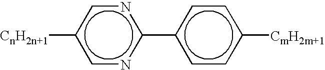 | 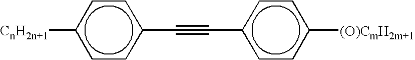 |
| PYP-nm | PTP-n(O)m |

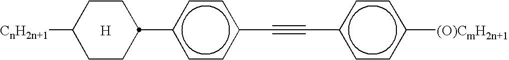

CPTP-n(O)m

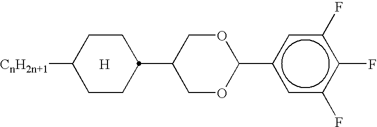

CDU-n-F

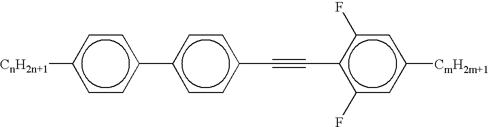

PPTUI-n-m

| | |
|---|---|
| 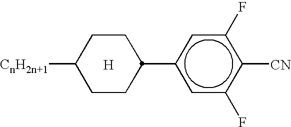 | 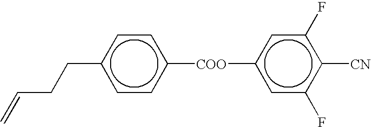 |
| PCH-nN.F.F | PZU-V2-N |

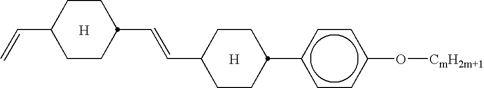

CVCP-V-Om

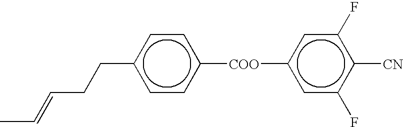

PZU-1V2-N

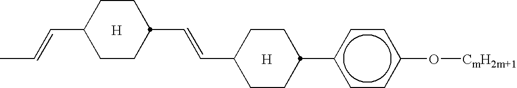

CVCP-1V-Om

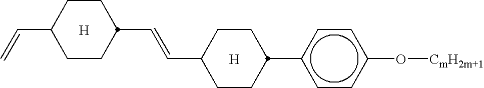

CVCP-V-m

| | |
|---|---|
| 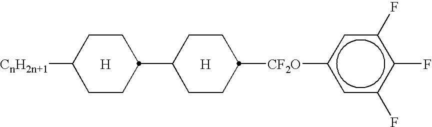 | 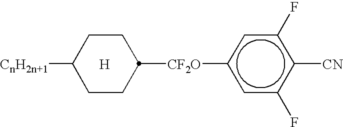 |
| CCQU-n-F | CQU-n-N |
| 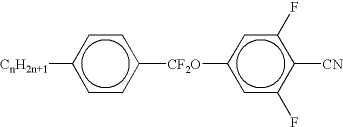 | 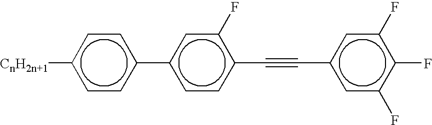 |
| PQU-n-N | PGTU-n-F |
| 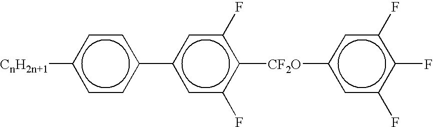 | 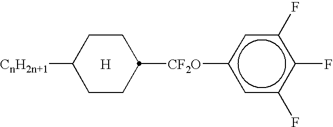 |
| PUQU-n-F | CQU-n-F |

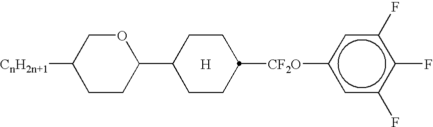

ACQU-n-F

TABLE C
Table C shows possible dopants which are generally added to the mixtures according to the invention.

| | |
|---|---|
| 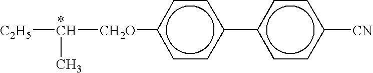 | 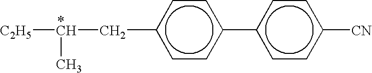 |
| C 15 | CB 15 |
| 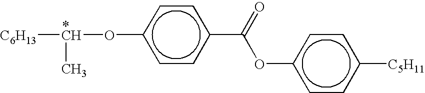 | 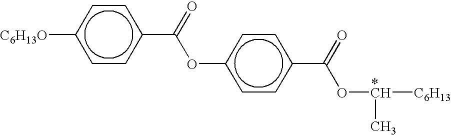 |
| CM 21 | R/S-811 |

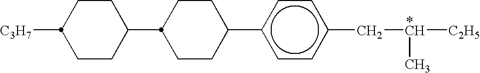

CM 44

| | |
|---|---|
| 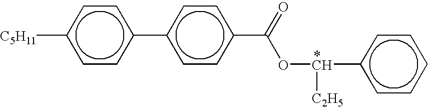 | 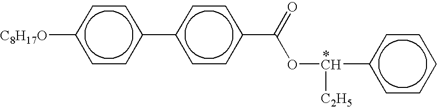 |
| CM 45 | CM 47 |

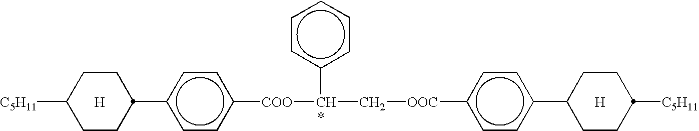

R/S-1011

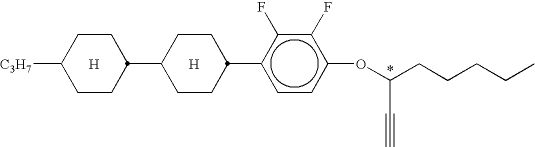

R/S-3011

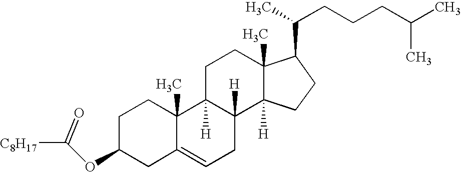

CN

| | |
|---|---|
| 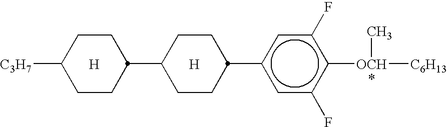 | 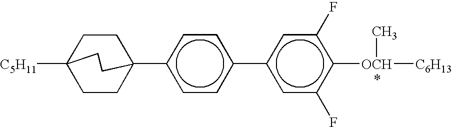 |
| R/S-2011 | R/S-4011 |

The following examples are intended to explain the invention without limiting it. Above and below, percentages are percent by weight. All temperatures are given in degrees Celsius. m.p. denotes melting point, cl.p.=clearing point. Furthermore, C=crystalline state, N=nematic phase, S=smectic phase and I=isotropic phase. The data between these symbols represent the transition temperatures. An denotes optical anisotropy (589 nm, 20° C.), and the flow viscosity ν₂₀ (mm²/sec) and the rotational viscosity γ₁ [mPa·s] were each determined at 20° C.

“Conventional work-up” means that water is added if necessary, the mixture is extracted with dichloromethane, diethyl ether, methyl tert-butyl ether or toluene, the phases are separated, the organic phase is dried and evaporated, and the product is purified by distillation under reduced pressure or crystallisation and/or chromatography. The following abbreviations are used:
DAST diethylaminosulfur trifluoride
DMEU 1,3-dimethyl-2-imidazolidinone
POT potassium tertiary-butoxide
THF tetrahydrofuran
pTsOH p-toluenesulfonic acid

### EXAMPLE 1

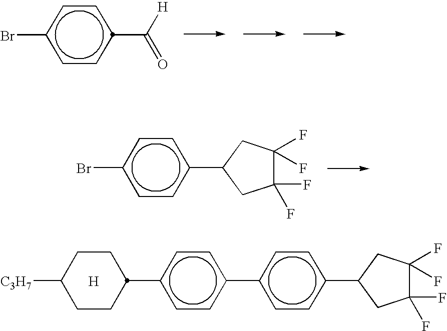 Step 1.1

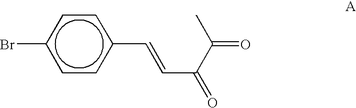

150 ml of 20% sodium hydroxide solution are added to a mixture of 0.195 mol of 4-bromobenzaldehyde and 0.185 mol of 3,3-dimethoxy-2-butanone in 200 ml of methanol, and the mixture is stirred at room temperature for 10 hours. The methanol is distilled off under reduced pressure, the residue is extracted with methyl tert-butyl ether, and the extract is dried over sodium sulfate and evaporated. The residue is distilled under reduced pressure. The distillate is stirred at room temperature for 15 hours with 10 g of p-toluenesulfonic acid and 1 l of acetone, the acetone is evaporated off, the residue is taken up in methyl tert-butyl ether, and the solution is washed with water until neutral. The solution is evaporated, and the residue is crystallised from pentane.
Step 1.2

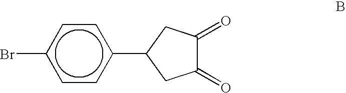

40 g of the dione A are dissolved in 400 ml of methanol and added dropwise to a solution of 20 g of magnesium methoxide in methanol at the boil. The mixture is boiled for 1 hour, the methanol is evaporated off, and the residue is acidified with dilute hydrochloric acid. The mixture is extracted with methyl tert-butyl ether, dried and evaporated. The residue is purified by recrystallisation.
Step 1.3

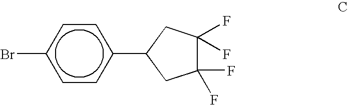

0.1 mol of bromophenylcyclopentadione B is reacted with 0.7 mol of SF₄ at 50° C. for 12 hours in an autoclave. After the mixture has cooled to room temperature and the reaction gases have been released, the reaction mixture is taken up in dichloromethane and washed until neutral, and the nonpolar product is separated off by means of column chromatography.
Step 1.4

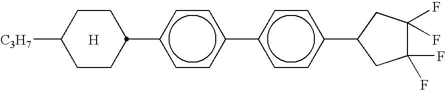

0.1 mol of 4-bromophenyltetrafluorocyclopentane C is dissolved in 200 ml of toluene with 0.1 mol of 4-propylcyclohexylphenylboronic acid, and 100 ml of 2 molar soda solution and 0.002 mol of Pd(PPh₃)₄ catalyst are added. The mixture is refluxed for 12 hours and finally subjected to conventional work-up.

The following compounds of the formula

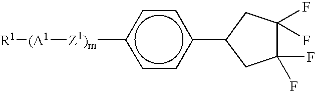

are prepared analogously:

| R¹ | —(A¹-Z¹)ₘ— |
|---|---|
| CH₃ | 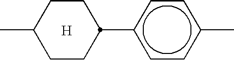 |
| C₂H₅ | 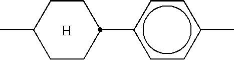 |
| n-C₄H₉ | 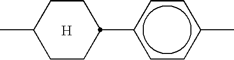 |
| n-C₅H₁₁ | 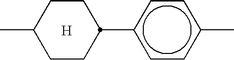 |
| n-C₆H₁₃ | 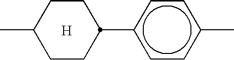 |
| n-C₇H₁₅ | 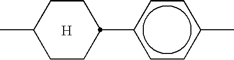 |
| CH₂═CH | 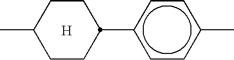 |
| CH₃CH═CH | 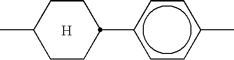 |
| CH₂═CHCH₂CH₂ | 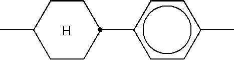 |
| CH₃CH═CHCH₂CH₂ | 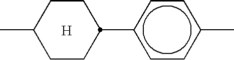 |
| CH₃ | 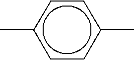 |
| C₂H₅ | 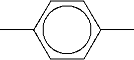 |
| n-C₃H₇ | 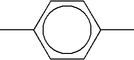 |
| n-C₄H₉ | 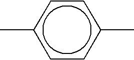 |
| n-C₅H₁₁ | 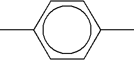 |
| n-C₆H₁₃ | 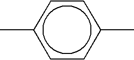 |

1. 3,3,4,4-Tetrafluorocyclopentane compounds of the formula I 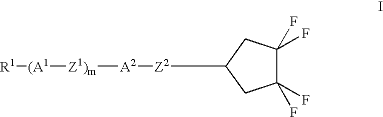
in which
R¹ is H, or an alkyl radical having up to 15 carbon atoms which is unsubstituted, monosubstituted by CN or CF₃ or at least monosubstituted by halogen, where, in addition, one or more CH₂ groups in these radicals may each, independently of one another, be replaced by —O—, —CH═CH—, 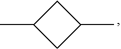
—CO—, —CO—O—, —O—CO— or —O—CO—O— in such a way that O atoms are not linked directly to one another,
A¹ and A² are each, independently of one another,
(a) a trans-1,4-cyclohexylene radical, in which, in addition, one or more non-adjacent CH₂ groups may be replaced by —O— and/or —S—,
(b) a 1,4-phenylene radical, in which, in addition, one or two CH groups may be replaced by N,
(c) 1,4-cyclohexenylene,
(d) a radical from the group consisting of 1,4-bicyclo[2.2.2]octylene, piperidine-1,4-diyl, naphthalene-2,6-diyl, decahydronaphthalene-2,6-diyl and 1,2,3,4-tetrahydronaphthalene-2,6-diyl,
where the radicals (a) to (d) may be substituted by one or more fluorine atoms,
Z¹ and Z² are each, independently of one another, —CO—O—, —O—CO—, —CH₂O—, —OCH₂—, —CH₂CH₂—, —CH═CH—, —C≡C—, —(CH₂)₄—, —CF₂O—, —OCF₂—, —C₂F₄—, —CH═CH—CH₂CH₂— or a single bond, and
m is 0, 1 or 2.

2. Compounds of the formulae I1-I30

| 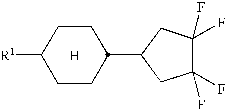 | I1 |
|---|---|
| 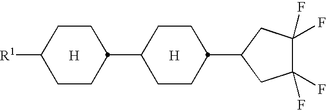 | I2 |
| 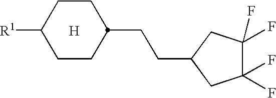 | I3 |
| 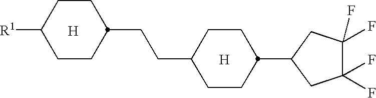 | I4 |
| 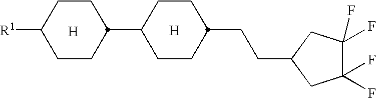 | I5 |
| 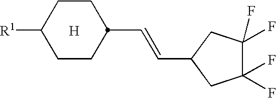 | I6 |
| 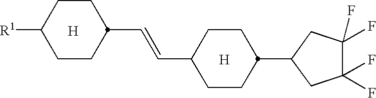 | I7 |
| 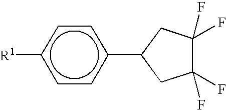 | I8 |
| 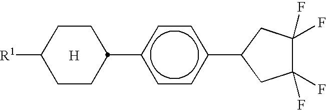 | I9 |
| 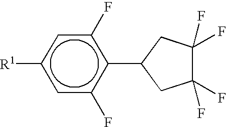 | I10 |
| 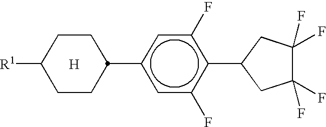 | I11 |
| 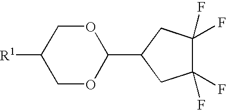 | I12 |
| 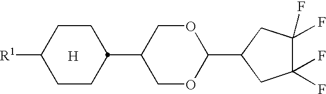 | I13 |
| 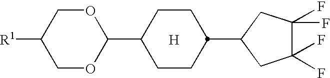 | I14 |
| 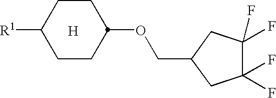 | I15 |
| 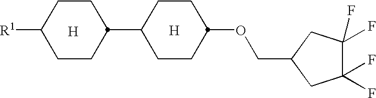 | I16 |
| 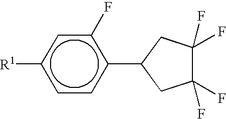 | I17 |
| 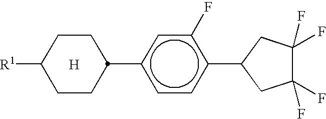 | I18 |
| 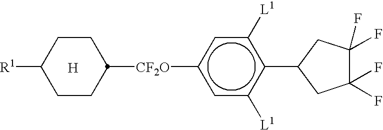 | I19 |
| 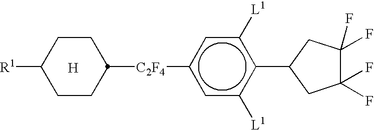 | I20 |
| 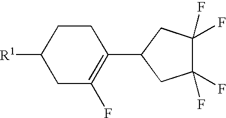 | I21 |
| 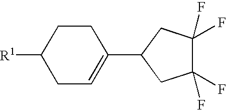 | I22 |
| 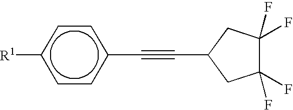 | I23 |
| 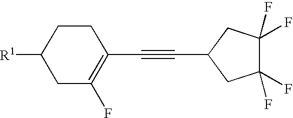 | I24 |
| 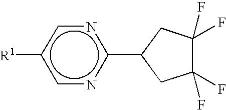 | I25 |
| 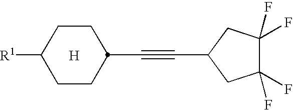 | I26 |
| 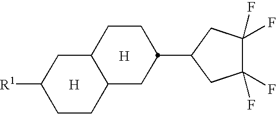 | I27 |
| 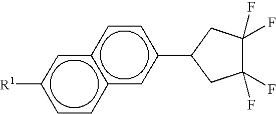 | I28 |
| 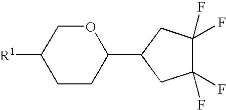 | 29 |
| 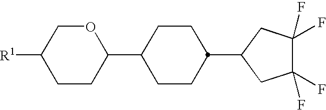 | 30 |

in which R¹ is as defined in claim 1, and L¹ and L² are each, independently of one another, H or F.

3. Compounds according to claim 1, characterised in that R¹ is alkyl, alkoxy, alkenyl or alkenyloxy having up to 7 carbon atoms.

4. Use of compounds of the formula I as components of liquid-crystalline media.

5. Liquid-crystalline medium having at least two liquid-crystalline components, characterised in that it comprises at least one compound of the formula I.

6. Liquid-crystal display element, characterised in that it contains a liquid-crystalline medium according to claim 5.

7. Electro-optical display element, characterised in that it contains, as dielectric, a liquid-crystalline medium according to claim 5.
